(19) United States
(12) Patent Application Publication
(10) Pub. No.: US 2003/0208095 A1
(43) Pub. Date: Nov. 6, 2003

(54) Particulate supports for oxidative dehydrogenation

(76) Inventors: Lisa M. Budin, Ponca City, OK (US); Joe D. Allison, Ponca City, OK (US); Sriram Ramani, Ponca City, OK (US)

Correspondence Address: CONLEY ROSE, P.C., P. O. BOX 3267, HOUSTON, TX, 77253-3267, US

(21) Appl. No.: 10/139,484

(22) Filed: May 6, 2002

(51) Int. Cl. C07C 5/373

(52) U.S. Cl. 585/658

(57) ABSTRACT

A catalyst useful for the production of olefins from alkanes via oxidative dehydrogenation (ODH) is disclosed. In accordance with a preferred embodiment of the present invention, a catalyst for use in ODH processes includes a base metal, a promoter metal, and a support comprising a plurality of discrete structures. A base metal is herein defined as a non-Group VIII metal, with the exception of iron, cobalt and nickel. Suitable base metals include Group IB-VIIB metals, Group IIIA-VA metals, Lanthanide metals, iron, cobalt and nickel. Suitable promoter metals include Group VIII metals (i.e. platinum, palladium, ruthenium, rhodium, osmium, and iridium). In some embodiments the support is fabricated from a refractory material. Suitable refractory support materials include alumina, stabilized aluminas, zirconia, stabilized zirconias (PSZ), titania, yttria, silica, niobia, and vanadia.

### STATEMENT REGARDING FEDERALLY SPONSORED RESEARCH OR DEVELOPMENT

Not applicable.

### CROSS-REFERENCE TO RELATED APPLICATIONS

Not applicable.

### FIELD OF THE INVENTION

This invention relates to oxidative dehydrogenation catalyst compositions and a method of using such catalysts in the presence of hydrocarbons. More particularly this invention relates to compositions of supported catalysts for the production of olefins by oxidative dehydrogenation of hydrocarbons in short-contact time reactors (SCTRs).

### BACKGROUND OF THE INVENTION

Dehydrogenation of hydrocarbons is an important commercial process. Dehydrogenation is the process used to convert aliphatics to olefins, mono-olefins to di-olefins, cycloalkanes to aromatics, alcohols to aldehydes and ketones, aliphatics and olefins to oxygenates, etc., by removing hydrogen chemically. In more practical terms, this process is responsible for products such as detergents, gasolines, pharmaceuticals, plastics, polymers, synthetic rubbers and many others. In addition, there is significant commercial use of the process for making many of the precursors for the above-mentioned products. For example, polyethylene is made from ethylene, which is made from the dehydrogenation of ethane (i.e. aliphatic to olefin). More ethylene is produced in the U.S. than any other organic chemical. Thus, it is easy to appreciate the significance of the dehydrogenation process to industry.

Traditionally, the dehydrogenation of hydrocarbons has been carried out using steam cracking or non-oxidative dehydrogenation processes. Thermal or steam cracking is an extremely energy intensive process that requires temperatures in excess of 800° C. About 1.4×10¹⁵ BTU's (equivalent to burning 1.6 trillion ft³ of natural gas) are consumed annually to produce ethylene. In addition, much of the reactant (ethane) is lost as coke deposition. Non-oxidative dehydrogenation is dehydrogenation whereby no molecular oxygen is added.

Oxidative dehydrogenation of hydrocarbons (ODH) with short contact time reactors is an alternative to traditional steam cracking and non-oxidative dehydrogenation processes. During an ODH reaction, oxygen is co-fed with saturated hydrocarbons balanced with an inert gas at a gas hourly space velocity (GHSV) of about 50,000 to 1,000,000 hr⁻¹. The oxygen may be fed as pure oxygen, air, oxygen-enriched air, oxygen mixed with a diluent, and so forth. Oxygen in the desired amount may be added in the feed to the dehydrogenation zone and oxygen may also be added in increments to the dehydrogenation zone. The contact time of the reactants with the catalyst is typically in the 10 to 200 ms range. At 1 bar pressure with monolith-supported catalysts, the reaction temperature is typically between 800-1100° C.

The capital costs for olefin production via ODH are significantly less than with the traditional processes, because ODH uses simple fixed bed reactor designs and high volume throughput. In addition, ODH is an autothermal process and requires no or very little energy to initiate the reaction. Energy savings over traditional, endothermal processes can be significant if the heat produced with ODH is recaptured and recycled. Often, the trade-off for saving money in commercial processes is loss of yield or selectivity, however, the ODH reactions are comparable to steam cracking in selectivity and conversion.

The benefits of ODH are not new. ODH processes have been studied on the laboratory scale for some time. The conventional ODH reactions involve the use of platinum-and-chromium containing catalysts.

Platinum and chromium oxide-based monolith catalysts were used for ethylene production with SCTRs in U.S. Pat. No. 6,072,097 and WO Pub. No. 00/43336, respectively. The monolith used in these catalysts were ceramic domes with 20-100 pores per linear inch. The domes were comprised of Al₂O₃, SiO₂, Mg-stabilized ZrO₂ (PSZ) or Y-stabilized ZrO₂ (YSZ). Ethylene yield with these reactors was about 50-55%.

U.S. Pat. No. 6,072,097 describes the use of Pt-coated monolith catalysts for ODH reactions in SCTRs. Pt in the range of 0.2-10% total weight of catalyst was claimed effective for ODH. Further impregnation of Sn or Cu on the Pt-coated surface (at Sn:Pt or Cu:Pt ratios of 0.5:1-7:1) promoted the ODH reactions. The light-off temperature with this type of catalysts was about 220° C., with reduced or no preheat after the light-off procedure. Light-off temperature is herein defined as the minimum temperature of the gases entering the catalyst zone at which the catalyst reaches a chemically active state so as to initiate a self-sustaining reaction between hydrocarbon(s) and oxygen (or oxygen containing gas), as indicated by an increase in the temperature of the gases exiting the catalyst zone.

WO Patent No. 0043336 describes the use of Cr, Cu, Mn or their mixed oxide-loaded monolith as the primary ODH catalysts promoted with less than 0.1% Pt. In addition, small amounts of Mn, Mg, Ni, Fe and Ag were used as promoters. Light-off temperature with these catalysts was about 350° C., with or without reduced preheat after the light-off procedure.

Despite a vast amount of research effort in this field, there is still a great need to identify effective catalyst systems for olefin synthesis, so as to maximize the value of the olefins produced and thus maximize the process economics. In addition, to ensure successful operation on a commercial scale, the ODH process must be able to achieve a high conversion of the hydrocarbon feedstock at high gas hourly space velocities, while maintaining high selectivity of the process to the desired products.

### SUMMARY OF THE INVENTION

In order to operate at very high flow rates, high pressure and using short contact time reactors, catalysts should be highly active, have excellent mechanical strength, resistance to rapid temperature fluctuations and thermal stability at oxidative dehydrogenation reaction temperatures.

The present invention provides a catalyst system for use in ODH that allows high conversion of the hydrocarbon feedstock at high gas hourly space velocities, while maintaining high selectivity of the process to the desired products. For the purposes of this disclosure, all listed metals are identified using the CAS naming convention.

In accordance with a preferred embodiment of the present invention, a catalyst for use in ODH processes includes a base metal, a promoter metal, and a support comprising a plurality of discrete structures. A base metal is herein defined as a non-Group VIII metal, with the exception of iron, cobalt and nickel. Suitable base metals include Group IB-VIIB metals, Group IIIA-VA metals, Lanthanide metals, iron, cobalt and nickel. Suitable promoter metals include Group VIII metals (i.e. platinum, palladium, ruthenium, rhodium, osmium, and iridium). In some embodiments the support is fabricated from a refractory material. Suitable refractory support materials include alumina, stabilized aluminas, zirconia, stabilized zirconias (PSZ), titania, yttria, silica, niobia, and vanadia.

In accordance with another preferred embodiment of the present invention, a method for converting gaseous hydrocarbons to olefins includes contacting a preheated alkane and oxygen stream with a catalyst containing a base metal, a promoter metal, and a support comprising a plurality of discrete structures, sufficient to initiate the oxidative dehydrogenation of the alkane (the preheat temperature being between 75° C. and 800° C.), maintaining a contact time of the alkane with the catalyst for less than 200 milliseconds, and maintaining oxidative dehydrogenation favorable conditions.

These and other embodiments, features and advantages of the present invention will become apparent with reference to the following description.

### DETAILED DESCRIPTION OF THE PREFERRED EMBODIMENTS

A new family of oxidative dehydrogenation catalysts having a base metal, a promoter metal, and a support comprising a plurality of discrete structures, or a particulate support, is described in the following representative examples. These catalysts are capable of catalytically converting C₁-C₁₀ hydrocarbons to olefins. They are preferably supported on any of various three-dimensional structures such as particulates including, but not limited to, balls, extrudates, powders, pills, and pellets. The inventors demonstrate that new particulate structures, when prepared as described in the following examples, are highly active oxidative dehydrogenation catalysts with sufficient mechanical strength to withstand high pressures and temperatures and permit a high flow rate of reactant and product gases when employed on-stream in a short contact time reactor for olefin production. Without wishing to be restricted to a particular theory, the inventors believe that the high surface area of the particulate-shaped catalysts provide improved heat and mass transfer in the catalytic reaction zone. Additionally, it is believed that the particulate-shaped catalysts provide ease of loading, decreased gas channeling, increased mechanical and thermal strength, and overall flexibility in catalyst design, as compared to conventional monolithic catalysts.

In some embodiments, Group VIII promoters and base metals are placed on refractory supports and used as catalysts for converting alkanes to alkenes via ODH. In a preferred embodiment of the present invention, light alkanes and O₂ are converted to the corresponding alkenes using novel promoted base metal catalysts.

Catalysts

The present catalysts preferably include a base metal, a Group VIII promoter metal, and a support comprising a plurality of discrete structures. Suitable base metals include Group IB-VIIB metals, Group IIIA-VA metals, Lanthanide metals, iron, cobalt and nickel. In some embodiments the support is fabricated from a refractory material. Suitable refractory support materials include alumina, stabilized aluminas, zirconia, stabilized zirconias (PSZ), titania, yttria, silica, niobia, and vanadia. In a preferred embodiment, the support is alumina, zirconia, or a combination thereof.

The present catalysts are preferably provided in the form of a plurality of distinct or discrete structures or particulates. The terms “distinct” or “discrete” structures or units, as used herein, refer to nonmonolithic supports in the form of divided materials such as granules, beads, pills, pellets, cylinders, trilobes, extrudates, spheres or other rounded shapes, or other manufactured configurations. Alternatively, the particulate material may be in the form of irregularly shaped particles. Preferably at least a majority (i.e., >50%) of the particles or distinct structures have a maximum characteristic length (i.e., longest dimension) of less than six millimeters, preferably less than three millimeters, and most preferably less than 1.5 millimeters. While the catalytic materials can be self-supporting, they are preferably provided as a surface layer on a particulate support.

In a preferred embodiment, the catalyst supports are coated with active metal components such as Group VIII promoters, base metals, and any combinations thereof. The coating may be achieved by any of a variety of methods known in the art, such as physical vapor deposition, chemical vapor deposition, electrolysis metal deposition, electroplating, melt impregnation, and chemical salt impregnation, followed by reduction.

Preferred catalyst systems in accordance with the present invention include Pt- or Pd-promoted Cr, Sn, Mn or Au metals supported on alumina granules or spheres. A more preferred catalyst system is Pt-promoted Cr supported on 35-50 mesh Alumina granules (see Examples).

Preferably, a millisecond contact time reactor, such as are known and described in the art, is used. By way of example only, operation of a millisecond contact time reactor is disclosed in detail in co-owned and co-pending U.S. patent Ser. No. 09/688,571, filed Oct. 16, 2000 and entitled “Metal Carbide Catalysts and Process for Producing Synthesis Gas,” which is incorporated herein by reference in its entirety. Use of a millisecond contact time reactor for the commercial scale conversion of light alkanes to corresponding alkenes will reduce capital investment and increase alkene production significantly. It has been discovered that an ethylene yield of 59% or higher in a single pass through the catalyst bed is achievable. This technology has the potential to achieve yields above those of the conventional technology at a much lower cost. The need for steam addition, as is currently required in the conventional cracking technology, is also eliminated by the present process. Nonetheless, in some embodiments of the present invention, the use of steam may be preferred. There is minimal coking in the present process and therefore little unit down time and loss of valuable hydrocarbon feedstock. Furthermore, the present novel catalysts improve the yield of the process to the desired alkene by 5% at atmospheric pressure and 3-7 standard liters per minute (SLPM) flowrate conditions.

In some embodiments, ODH is carried out using the hydrocarbon feed mixed with an appropriate oxidant and possibly steam. Appropriate oxidants may include, but are not limited to air, oxygen-enriched air, I₂, O₂, N₂O and SO₂. Use of the oxidant prevents coke deposition and aids in maintaining the reaction. Steam, on the other hand, may be used to activate the catalyst, remove coke from the catalyst, or serve as a diluent for temperature control.

Without further elaboration, it is believed that one skilled in the art can, using the description herein, utilize the present invention to its fullest extent. The following Examples are to be construed as illustrative, and not as limiting the disclosure in any way whatsoever.

### EXAMPLES

In the following examples, the supports were purchased from Sud-Chemie or NorPro Corporation. In a first layer, the base metal coatings were added by an incipient wetness technique, wherein incipient wetness of the supports was achieved using aqueous solutions of a soluble metal salts such as nitrate, acetate, chlorides, acetylacetonate or the like. In a second layer, the Group VIII promoter coatings were similarly added by an incipient wetness technique. For higher metal loading, the process may be repeated until desired loading is achieved, with intermediate calcination after adding the aqueous solutions of the catalytic metals.

While the following examples were prepared by an incipient wetness technique, any technique known to those skilled in the art may be alternatively used. The final catalysts tested were in the form of 1/16″-1/10″ spheres or 35-50 mesh granules, with an operating pressure approximately equal to atmospheric pressure. Results are shown below in Table 1.

TABLE 1

| | Catalyst | Amount | | | Ethane/Oxygen | | | | | |
|---|---|---|---|---|---|---|---|---|---|---|
| | (metals in | of | Preheat | Total | molar | Catalyst | % | % | | % |
| | wt % of | Catalyst | Temp | Flowrate | ratio (10% N₂ | Temp | Ethane | Oxygen | % C₂H₄ | C₂H₄ |
| Ex. | catalyst) | (g) | (° C.) | (GHSV, h⁻¹) | dilution used) | (° C.) | Conv. | Conv. | selectivity | yield |
| A | 0.05% Pt, | 0.4 | 350 | 430,300 | 2.1 | 904 | 83.3 | 97.6 | 71.3 | 59.4 |
| | 2.7% Cr | | | 717,200 | 2.1 | 910 | 83.8 | 97.3 | 65.7 | 55.0 |
| | on 35-50 | | | 1,004,100 | 2.1 | 914 | 81.1 | 96.0 | 64.5 | 52.3 |
| | mesh | 0.8 | 350 | 223,800 | 2.1 | 891 | 83.1 | 98.7 | 64.8 | 53.9 |
| | Alumina | | | 372,950 | 2.1 | 921 | 84.0 | 98.2 | 62.5 | 52.6 |
| | granules | 1.9 | 350 | 148,000 | 2.1 | 895 | 84.8 | 98.8 | 56.2 | 47.6 |
| B | 2% Pt, | 0.8 | 350 | 147,200 | 1.8 | 952 | 91.6 | 98.3 | 57.9 | 53.0 |
| | 0.4% Au | | | 147,200 | 2.1 | 916 | 79.8 | 97.0 | 64.9 | 52.0 |
| | on 1/10″ | | | 245,300 | 1.8 | 978 | 92.6 | 98.1 | 55.3 | 51.2 |
| | Alumina | 1.9 | | 75,000 | 1.8 | 918 | 89.5 | 98.1 | 59.2 | 53.0 |
| | spheres | | | 124,300 | 1.8 | 955 | 91.5 | 98.3 | 55.7 | 51.0 |
| C | 0.1% Pt, | 2 | 350 | 248,600 | 1.9 | 919 | 91.5 | 98.4 | 59.1 | 54.0 |
| | 1.5% Sn | | | 248,600 | 2.1 | 903 | 85.0 | 97.4 | 63.7 | 54.1 |
| | on 1/16″ | | | 248,600 | 2.4 | 866 | 73.1 | 96.4 | 67.5 | 49.3 |
| | Alumina | | 300 | 248,600 | 2.1 | 876 | 81.6 | 97.2 | 64.9 | 53.0 |
| | spheres | | | | | | | | | |
| D | 0.5% Pt, | 2 | 300 | 164,100 | 2.0 | 862 | 85.7 | 98.8 | 65.2 | 55.9 |
| | 1.5% Sn | | | 248,600 | 2.0 | 892 | 87.7 | 99.0 | 62.2 | 54.6 |
| | on 1/16″ | | | | | | | | | |
| | Alumina | | | | | | | | | |
| | spheres | | | | | | | | | |

From Example A, it can be seen that as the amount of catalyst decreases at a constant gas flowrate of 5 SLPM and Fuel/Oxygen ratio of 2.1, ethylene yield increases from 47.6% to 55.0%, indicating that these conditions promote the short contact time ODH reaction. Without wishing to be bound by any specific theory, the inventors believe that this improved performance appears to be a function of weight hourly space velocity (WHSV). On the other hand, at a constant catalyst weight of 0.4 gram, an increase of gas flowrate (i.e., GHSV) results in a decrease of ethylene yield from 59.4% to 52.3%. However, this decrease was smaller when 0.8 gram of catalyst was used. It is believed that combining the optimum catalyst weight and flowrates would result in higher ethylene yields than reported here.

From Example C, it can be seen that as the fuel/oxygen ratio increases, ethane and oxygen conversions decrease and ethylene selectivity increases. For this case, a ratio of 2:1 appears to be optimal, but it must be noted that this is a function of other parameters such as flowrate and preheat temperature.

Comparing Examples C and D, the increased Pt loading in Example D appears to result in slightly higher ethylene yield. Overall, these examples illustrate the improved ethylene yields that can be achieved by using particulate supports for ODH catalysts. Without wishing to be bound by any theory, it is believed that the significantly higher ethylene yields seen with Example A, even though the Pt loading was low, could be due to the higher surface area and smaller particle size of the granular support. The results indicate that further optimization of the support structure, catalyst composition and process variables would lead to improved ethylene yields.

Process Conditions

Any suitable reaction regime can be applied in order to contact the reactants with the present catalyst. One suitable regime is a fixed bed reaction regime, in which the catalyst is retained within a reaction zone in a fixed arrangement. Catalysts may be employed in the fixed bed regime, using fixed bed reaction techniques well known in the art. Preferably a millisecond contact time reactor is employed. Several schemes for carrying out oxidative dehydrogenation of hydrocarbons in a short contact time reactor have been described in the literature and one of ordinary skill in the art will understand the operation of short contact time reactors and the applicability of the present invention thereto.

Accordingly, a feed stream comprising a hydrocarbon feedstock and an oxygen-containing gas is contacted with one of the above-described catalysts in a reaction zone maintained at conversion-promoting conditions effective to produce an effluent stream comprising alkenes. The hydrocarbon feedstock may be any gaseous hydrocarbon having a low boiling point, such as ethane, natural gas, associated gas, or other sources of light hydrocarbons having from 1 to 10 carbon atoms. In addition, hydrocarbon feeds including naphtha and similar feeds may be employed. The hydrocarbon feedstock may be a gas arising from naturally occurring reserves of ethane. Preferably, the feed comprises at least 50% by volume alkanes (<C₁₀).

The hydrocarbon feedstock is contacted with the catalyst as a gaseous phase mixture with an oxygen-containing gas, preferably pure oxygen. The oxygen-containing gas may also comprise steam and/or methane in addition to oxygen. Alternatively, the hydrocarbon feedstock is contacted with the catalyst as a mixture with a gas comprising steam and/or methane.

The process is operated at atmospheric or superatmospheric pressures, the latter being preferred. The pressures may be from about 80 kPa to about 32,500 kPa, preferably from about 130 kPa to about 5,000 kPa. The preheat temperature of the present invention occurs at temperatures of from about 75° C. to about 800° C., preferably from about 150° C. to about 700° C., and most preferably from 150° C. to about 350° C. when an alumina granular or spherical support with metal loading is used. The hydrocarbon feedstock and the oxygen-containing gas are preferably pre-heated before contact with the catalyst. The hydrocarbon feedstock and the oxygen-containing gas are passed over the catalyst at any of a variety of space velocities.

Gas hourly space velocities (GHSV) for the present process, stated as normal liters of gas per kilogram of catalyst per hour, are from about 20,000 to at least about 100,000,000 hr⁻¹, preferably from about 50,000 to about 1,000,000 hr⁻¹. Preferably the catalyst is employed in a millisecond contact time reactor. The process preferably includes maintaining a catalyst residence time of no more than 200 milliseconds for the reactant gas mixture. Residence time is inversely proportional to space velocity, and high space velocity indicates low residence time on the catalyst. An effluent stream of product gases, including alkenes, CO, CO₂, H₂, H₂O, and unconverted alkanes emerge from the reactor.

In some embodiments, unconverted alkanes may be separated from the effluent stream of product gases and recycled back into the feed.

In some embodiments the use of steam may be employed. As mentioned above, steam may be used to activate the catalyst, remove coke from the catalyst, or serve as a diluent for temperature control.

While the preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. For example, the present invention may be incorporated into a gas to liquids plant (GTL) or may stand alone. Accordingly, the scope of protection is not limited by the description set out above, but is only limited by the claims which follow, that scope including all equivalents of the subject matter of the claims. The disclosures of all patents and publications cited herein are incorporated by reference in their entireties.

What is claimed is:

1. A catalyst for use in oxidative dehydrogenation processes comprising:
a base metal;
a promoter metal; and
a support comprising a plurality of discrete structures,
wherein said base metal and promoter metal are coated on said support.

2. The catalyst of claim 1 wherein the discrete structures are particulates.

3. The catalyst of claim 2 wherein the plurality of discrete structures comprises at least one geometry chosen from the group consisting of powders, particles, granules, spheres, beads, pills, rings, pellets, balls, noodles, cylinders, extrudates and trilobes.

4. The catalyst of claim 1 wherein at least a majority of the discrete structures each have a maximum characteristic length of less than six millimeters.

5. The catalyst of claim 4 wherein the majority of the discrete structures each have a maximum characteristic length of less than about three millimeters.

6. The catalyst of claim 1 wherein the support is selected from the group consisting of alumina, stabilized aluminas, zirconia, stabilized zirconias (PSZ), titania, yttria, silica, niobia, and vanadia.

7. The catalyst of claim 6 wherein the support comprises alumina, zirconia, or a combination thereof.

8. The catalyst of claim 1 wherein the base metal is selected from the group consisting of Group IB-VIIB metals, Group IIIA-VA metals, Lanthanide metals, iron, cobalt or nickel.

9. The catalyst of claim 8 wherein the base metal is Cr.

10. The catalyst of claim 8 wherein the preheat temperature is below 700° C.

11. The catalyst of claim 1 wherein the promoter metal is selected from the group consisting of Ru, Rh, Pd, Pt, Os, and Ir.

12. The catalyst of claim 11 wherein the promoter metal loading is less than 3% the total weight of the catalyst.

13. The catalyst of claim 11 wherein the promoter metal is Pt.

14. The catalyst of claim 11 wherein the preheat temperature is below 350° C.

15. A method for converting gaseous hydrocarbons to olefins comprising:
heating a feed stream comprising an alkane and an oxidant to a temperature of approximately 75° C. to 800° C.;
contacting the feed stream with a catalyst comprising a base metal, a promoter metal, and support comprising a plurality of discrete structures;
maintaining a contact time of the alkane with the catalyst for less than 200 milliseconds; and
maintaining oxidative dehydrogenation favorable conditions.

16. The method of claim 15 wherein the oxidant comprises an oxygen containing gas.

17. The method of claim 16 wherein the oxidant is essentially pure oxygen.

18. The method of claim 15 wherein the feed stream is heated to a temperature below 700° C.

19. The method of claim 15 wherein the feed stream is heated to a temperature below 350° C.

20. The catalyst of claim 15 wherein at least a majority of the discrete structures each have a maximum characteristic length of less than six millimeters.

21. The catalyst of claim 20 wherein the majority of the discrete structures each have a maximum characteristic length of less than about three millimeters.

22. The catalyst of claim 15 wherein the support is selected from the group consisting of alumina, stabilized aluminas, zirconia, stabilized zirconias (PSZ), titania, yttria, silica, niobia, and vanadia.

23. The method of claim 15 wherein the feed stream is contacted with the catalyst at a gas hourly space velocity of at least 20,000 hr⁻¹.

24. The method of claim 15 wherein the feed stream is contacted with the catalyst at a gas hourly space velocity up to 100,000,000 hr⁻¹.

25. The method of claim 15 wherein the feed stream is maintained at a pressure in excess of 80 kPa while contacting the catalyst.

26. The method of claim 25 wherein the pressure is up to about 32,500 kPa.

27. The method of claim 25 wherein the pressure is between 130-5,000 kPa.

28. The method of claim 15 wherein the contact time of the alkane and catalyst is less than 50 milliseconds.

29. An oxidative dehydrogenation catalyst comprising a base metal, a promoter metal, and a support comprising a plurality of discrete structures.
